(19) United States
(12) Patent Application Publication
(10) Pub. No.: US 2026/0306757 A1
(43) Pub. Date: Oct. 8, 2026

(54) AN INSUFFLATOR, AN INSUFFLATING SYSTEM AND A METHOD FOR INSUFFLATING A CAVITY IN THE BODY OF A HUMAN OR ANIMAL SUBJECT

(71) Applicant: Palliare Limited, Dangan, Galway (IE)

(72) Inventor: John O'DEA, Bearna (IE)

(73) Assignee: Palliare Limited, Dangan, Galway (IE)

(21) Appl. No.: 19/481,916

(22) PCT Filed: May 8, 2024
(86) PCT No.: PCT/IE2024/000004

§ 371 Date: Nov. 6, 2025

(30) Foreign Application Priority Data
May 8, 2023 (IE) S2023/0142
Aug. 23, 2023 (IE) S2023/0344

(51) Int. Cl. A61M 13/00; A61B 1/00; A61B 1/018; A61B 1/273

(52) U.S. Cl. CPC A61M 13/003; A61B 1/00119; A61B 1/00128; A61B 1/018; A61B 1/273; A61M 2202/0007; A61M 2202/02; A61M 2205/3327; A61M 2205/3344; A61M 2205/50; A61M 2205/7509; A61M 2205/7518; A61M 2206/10

(57) ABSTRACT

An insufflating system (1) comprises an insufflator (4) and an endoscope (5). The insufflator (4) comprises a flow control valve (17) for supplying insufflating gas to a cavity of a subject, and an isolating valve (22) for applying a vacuum to the cavity, both of which are connected to an instrument channel (8) of the endoscope (5) through a tube set (30). A pressure sensor (24) monitors cavity pressure'through the instrument channel (8). The insufflator (4) is operable in a first operating mode in which a microprocessor (21) operates the flow control valve (17) to maintain the cavity pressure at a set pressure, and on the cavity pressure exceeding a predefined upper pressure, the microprocessor (21) operates the isolating valve (22) for applying vacuum to the cavity, and for further second time periods until the cavity pressure returns to the set pressure. The insufflator (4) is operable in a second operating mode in which the microprocessor (21) alternately and sequentially operates the flow control valve (17) and the isolating valve (22) to apply sequential pressure/vacuum cycles to the cavity in order to withdraw smoke generated in the cavity, while maintaining the cavity pressure within plus or minus 1 mmHg or 2 mmHg of the set pressure.

The present invention relates to an insufflator for insufflating a cavity in the body of a human or animal subject, and the invention also relates to an insufflating system comprising an insufflator and an endoscope for insufflating a cavity in the body of a human or animal subject. Additionally, the invention relates to a method for insufflating a cavity in the body of a human or animal subject.

The term “cavity” is used in this specification, and the claims appended hereto to include any cavity, vessel, lumen or organ, and any cavity in any vessel, lumen or organ in the body of a human or animal subject which may be insufflated.

As surgical procedures have evolved, there has been an increasing move to perform procedures using an endoscope or a colonoscope. Hereinafter, through this specification and the claims appended hereto, the term “endoscope” is used to include an endoscope and a colonoscope. The key benefit of endoscopic procedures is that no incision has to be made on the skin of a patient, with the endoscope being introduced through a natural body orifice, such as the mouth or anus. Some basic endoscopic procedures, for example, removing small polyps in the colon, can be performed using endoscopic tools which pass through channels, generally, an instrument channel or channels within the endoscope. Other advanced procedures utilise overtubes through which the endoscope passes. These overtubes contain instrumentation and channels themselves, which are used in performing procedures, such as transoral incisionless fundoplication (TIF) procedures for treating gastroesophageal reflux from Endogastric Solutions. As these procedures evolve, so has the need to improve insufflating, namely, the introduction of gas to creating a working space for an endoscopist in the cavity in which the procedure is being carried out, and in particular, for the removal of smoke generated in the cavity in, for example, a low pressure dissecting procedure. In general, since only one channel is available in an endoscope for both delivery of insufflating gas to the cavity and the withdrawal of smoke and other gases from the cavity, the removal of smoke during a low pressure dissecting procedure or other such smoke generating procedures, is particularly challenging. Additionally, the removal of high pressure gases generated in the cavity or introduced into the cavity during an endoscope procedure, is also challenging.

There is therefore a need for an insufflator which addresses these problems, and there is also a need for an insufflating system which addresses these problems. Further, there is a need for a method for insufflating a cavity in the body of a human or animal subject which addresses these problems.

The present invention is directed towards providing such an insufflator, such an insufflating apparatus and such a method.

According to the invention there is provided an insufflator for insufflating a cavity in the body of a human or animal subject, the insufflator comprising a first control means for controlling the supply of insufflating gas to the cavity, a second control means for controlling application of a vacuum to the cavity, a pressure monitoring means for monitoring pressure in the cavity (cavity pressure) and for producing a signal indicative of the cavity pressure, and a signal processor configured to read the signal from the pressure monitoring means and to control the first and second controls means in response to the signal read from the pressure monitoring means, wherein the signal processor is configured to selectively operate the insufflator in selectable ones of a first operating mode and a second operating mode, and in the first operating mode the signal processor is configured to operate the first control means in response to the signal read from the pressure monitoring means to control the supply of insufflating gas to the cavity for maintaining the cavity pressure substantially at a set pressure, and on the signal read from the pressure monitoring means being indicative of the cavity pressure exceeding a predefined upper pressure greater than the set pressure, the signal processor is configured to operate the second control means to apply a vacuum to the cavity until the cavity pressure falls to the set pressure, or to apply a vacuum to the cavity for a first predefined time period, and in the second operating mode, the signal processor is configured to control the first and second control means to sequentially and alternately supply insufflating gas to the cavity and to apply a vacuum to the cavity in a plurality of sequential pressure/vacuum cycles.

In one embodiment of the invention the first predefined time period lies in the range of 1 second to 5 seconds, and preferably, in the range of 2 seconds to 4 seconds, and ideally, the first predefined time period is approximately 3 seconds.

In another embodiment of the invention the signal processor is responsive to failure of the cavity pressure falling to or below the set pressure at the end of the first predefined time period to operate the second control means to apply the vacuum to the cavity for at least one second time period.

In another embodiment of the invention the signal processor is responsive to failure of the cavity pressure falling to or below the set pressure at the end of each second time period to operate the second control means to apply the vacuum to the cavity for another second time period.

In another embodiment of the invention the signal processor is programmed to compute the time duration of the first one of the second time periods as a function of the difference between the set pressure and the cavity pressure at the end of the first predefined time period.

In another embodiment of the invention the signal processor is programmed to compute the time duration of the first one of the second time periods as a function of the rate at which the cavity pressure dropped during the first predefined time period.

In another embodiment of the invention the signal processor is programmed to compute the time duration of the second and each subsequent one of the second time periods as a function of the difference between the set pressure and the cavity pressure at the end of the previous one of the second time periods.

In a further embodiment of the invention the signal processor is programmed to compute the time duration of the second one and each subsequent one of the second time periods as a function of the rate at which the cavity pressure dropped during the previous one of the second time periods.

In one embodiment of the invention the time duration of each second time period is shorter than the time duration of the first predefined time period, and preferably, the time duration of each subsequent second time period is shorter than the time duration of the immediately previous second time period.

In an alternative embodiment of the invention the time duration of each one of the second time periods is the same as the time duration of the first predefined time period.

In another embodiment of the invention the signal processor is programmed to operate the second control means to terminate the application of the vacuum to the cavity at the end of each one of the second time periods, and to read the signal from the pressure monitoring means.

In another embodiment of the invention the signal processor is programmed to operate the second control means to terminate the application of the vacuum to the cavity at the end of the first predefined time period and to read the signal from the pressure monitoring means.

In another embodiment of the invention in the first operating mode of the insufflator the signal processor is responsive to the cavity pressure exceeding the predefined upper pressure to operate the first control means to terminate the supply of insufflating gas to the cavity until the cavity pressure falls to or below the set pressure.

In one embodiment of the invention the predefined upper pressure lies in the range of 0.5 mmHg to 5 mmHg above the set pressure, and preferably, the predefined upper pressure lies in the range of 0.5 mmHg to 4 mmHg above the set pressure, and preferably, in the range of 1 mmHg to 4 mmHg above the set pressure, although, in some embodiments of the invention the predefined upper pressure may be approximately 1 mmHg above the set pressure.

In one embodiment of the invention in the second operating mode, the signal processor is programmed to operate the first and second control means during each pressure/vacuum cycle to maintain the cavity pressure within a predefined pressure range about the set pressure.

In one embodiment of the invention the predefined pressure range about the set pressure lies in the range of 3 mmHg above the set pressure and 3 mmHg below the set pressure. Preferably, the predefined pressure range about the set pressure lies in the range of 2 mmHg above the set pressure and 2 mmHg below the set pressure. Advantageously, the predefined pressure range above the set pressure lies in the range of 1 mmHg above the set pressure and 1 mmHg below the set pressure.

In another embodiment of the invention in the second operating mode, the signal processor is programmed to operate the first control means to supply the insufflating gas to the cavity for a predefined gas supply time period in each pressure/vacuum cycle.

In another embodiment of the invention in the second operating mode, the signal processor is programmed to operate the second control means to apply vacuum to the cavity for a predefined vacuum application time period in each pressure/vacuum cycle.

Preferably, each predefined vacuum application time period lies in the range of 1 second to 5 seconds, and advantageously, each predefined vacuum application time period lies in the range of 2 seconds to 4 seconds, and ideally, each predefined vacuum application time period is approximately 3 seconds.

In another embodiment of the invention each predefined gas supply time period lies in the range of 4 seconds to 10 seconds, and preferably, each predefined gas supply time period lies in the range of 6 seconds to 8 seconds, and ideally, each predefined gas supply time period is approximately 7 seconds.

In another embodiment of the invention the duration of the predefined vacuum application time period of each pressure/vacuum cycle lies in the range of one eighth to one half of the duration of each pressure/vacuum cycle, and preferably, the duration of the predefined vacuum application time period is approximately one third of the duration of each pressure/vacuum cycle.

In one embodiment of the invention in the second operating mode the signal processor is programmed to operate the second control means to prevent vacuum being applied to the cavity during the predefined gas supply time period of each pressure/vacuum cycle, and preferably, the signal processor is programmed to operate the first control means to prevent insufflating gas being supplied to the cavity during the predefined vacuum application time period of each pressure/vacuum cycle.

In one embodiment of the invention in the first operating mode the signal processor is programmed to operate the second control means to prevent vacuum being applied to the cavity when the first control means is being operated to supply insufflating gas to the cavity

In another embodiment of the invention in the first operating mode the signal processor is programmed to operate the first control means to prevent insufflating gas being supplied to the cavity when the second control means is being operated to apply a vacuum to the cavity.

Preferably, in the first operating mode the signal processor is responsive to the signal from the pressure monitoring means being indicative of the cavity pressure falling to or below the set pressure to operate the first control means to supply insufflating gas to the cavity for maintaining the cavity pressure at the set pressure.

In one embodiment of the invention the signal processor is programmed to be responsive to the signal read from the pressure sensing means being indicative of the cavity pressure having fallen to a predefined lower pressure lower than the set pressure during operation of the second control means applying vacuum to the cavity, to operate the second control means to terminate application of vacuum to the cavity, and to operate the first control means to supply insufflating gas to the cavity until the cavity pressure returns to the set pressure.

In another embodiment of the invention the predefined lower pressure lies in the range of 2 mmHg to 10 mmHg below the set pressure. Preferably, the predefined lower pressure lies in the range of 2 mmHg to 8 mmHg below the set pressure. Advantageously, the predefined lower pressure lies is approximately 6 mmHg below the set pressure.

In one embodiment of the invention the set pressure lies in the range of 2 mmHg to 15 mmHg, and preferably, the set pressure lies in the range of 5 mmHg to 15 mmHg, and ideally, the set pressure is approximately 10 mmHg.

In another embodiment of the invention the set pressure is selectable.

In another embodiment of the invention the insufflator comprises a first outlet port communicating with the first control means for accommodating insufflating gas therethrough to the cavity.

In another embodiment of the invention the insufflator comprises a second outlet port communicating with the second control means through which vacuum is applied to the cavity.

In another embodiment of the invention the insufflator comprises a third port for communicating the pressure monitoring means with the cavity. Alternatively, the pressure monitoring means is connected to the first outlet port or to the second outlet port. In a further embodiment of the invention the pressure monitoring means is selectively connectable to the third port or to one or both of the first or second outlet ports.

In one embodiment of the invention the insufflator comprises an insufflating gas source communicating with the first control means. Preferably, the insufflating gas source comprises a pressurised container containing the pressurised insufflating gas.

In an alternative embodiment of the invention the insufflator comprises a first inlet port communicating with the first control means for receiving insufflating gas from an external insufflating gas source thereof.

Preferably, the first control means comprises a flow control valve or a flow controller.

In one embodiment of the invention the second control means comprises a vacuum pump,

In an alternative embodiment of the invention the insufflator comprises a second inlet port communicating with the second control means for coupling the insufflator to an external vacuum source.

In one embodiment of the invention the second control means comprises an isolating valve selectively and alternately operable in an isolating state isolating the second outlet port from the second inlet port, and in a communicating state communicating the second outlet port with the second inlet port for applying vacuum from the external vacuum source to the second outlet port.

In another embodiment of the invention the pressure monitoring means comprises a pressure sensor, and preferably, the pressure monitoring means is located in the insufflator.

In another embodiment of the invention an interface means is provided communicating with the signal processor for selecting and entering the operating mode of the insufflator, and preferably, the interface means comprises at least one button switch for selecting each one of the first and second operating modes.

In another embodiment of the invention the signal processor is programmed to operate the insufflator in the first operating mode as a default operating mode.

In one embodiment of the invention a filter is provided for filtering gases drawn from the cavity during application of a vacuum to the cavity. Preferably, the filter comprises a bacterial-viral filter, and advantageously, the filter comprises a second activated carbon filter layer.

In one embodiment of the invention the filter is located between the cavity and the insufflator.

In another embodiment of the invention a water collecting means is provided for collecting water entrained in gases drawn from the cavity during application of a vacuum to the cavity, and preferably, the water collecting means is located between the filter and the cavity.

In one embodiment of the invention the water collecting means comprises a water trap.

The invention also provides an insufflating system for insufflating a cavity in the body of a human or animal subject, the insufflating system comprising an insufflator comprising a first control means for controlling the supply of insufflating gas to a cavity in the body of a subject for insufflating the cavity to a set pressure, and a second control means for applying a vacuum to the cavity, an endoscope comprising a channel capable of accommodating an insufflating gas to the cavity of the subject, and a connecting means connecting the insufflator to the said channel of the endoscope for alternately applying insufflating gas and a vacuum to the cavity through the said channel of the endoscope.

In one embodiment of the invention the first control means and the second control means are configured for sequentially supplying insufflating gas and applying the vacuum to the said channel of the endoscope.

In another embodiment of the invention the first control means and the second control means are configured to supply insufflating gas and to apply the vacuum in sequential insufflating gas/vacuum cycles to the said channel of the endoscope.

Preferably, the first control means is configured for supplying insufflating gas to the said channel of the endoscope during each insufflating gas/vacuum cycle for a predefined gas supply time period, and the second control means is configured for applying the vacuum to the said channel of the endoscope during each insufflating gas/vacuum cycle for a predefined vacuum application time period.

In one embodiment of the invention the second control means is responsive to the cavity pressure exceeding the set pressure or a predefined upper pressure greater than the set pressure for applying the vacuum to the said channel of the endoscope.

In one embodiment of the invention the second control means is responsive to the cavity pressure exceeding the preset pressure or the predefined upper pressure for applying the vacuum to the said channel of the endoscope for one predefined vacuum application time period.

Preferably, the second control means is configured to apply the vacuum to the said channel of the endoscope until the cavity pressure is reduced to or below the set pressure or to a predefined lower pressure below the set pressure.

In one embodiment of the invention the connecting means comprises a first port being configured for communicating with the said channel of the endoscope, a second port being configured for communicating with the first control means, and a third port being configured for communicating with the second control means.

In another embodiment of the invention the connecting means comprises a Y-connector.

Preferably, the insufflator comprises a first outlet port and a second outlet port, and the first control means communicates with the first outlet port, and the second control means communicates with the second outlet port.

Advantageously, the second port of the connecting means is configured for communicating with the first outlet port, and the third port of the connecting means is configured for communicating with the second outlet port.

In one embodiment of the invention the insufflator comprises a pressure monitoring means adapted for selectively connecting to the said channel of the endoscope or to another channel of the endoscope for monitoring the pressure in the cavity.

In another embodiment of the invention the channel of the endoscope capable of accommodating insufflating gas to the cavity of a subject comprises one of an instrument channel of the endoscope, an insufflating channel of the endoscope, a vacuum channel of the endoscope, or a channel defined by a tubular member attached and extending to the endoscope externally thereof.

In one embodiment of the invention the vacuum applied to the cavity by the second control means is adapted for drawing smoke and/or gases from the cavity.

Preferably, the first control means is configured to apply insufflating gas to the cavity for maintaining the cavity pressure at the set pressure.

Further the invention provides an insufflating system for insufflating a cavity in the body of a human or animal subject, the insufflating system comprising the insufflator according to the invention, and an endoscope comprising a channel capable of accommodating an insufflating gas to the cavity of the subject, and a connecting means connecting the insufflator to the said channel for alternately applying the insufflating gas and the vacuum to the said channel of the endoscope.

In one embodiment of the invention the connecting means is adapted for connecting to the first control means and to the second control means and to the said channel of the endoscope.

In another embodiment of the invention the connecting means comprises a first port being configured for communicating with the said channel of the endoscope, a second port being configured for communicating with the first control means, and a third port configured for communicating with the second control means.

Preferably, the second port of the connecting means is adapted for coupling to the first outlet port of the insufflator, and the third port of the connecting means is adapted for coupling to the second outlet port of the insufflator.

In one embodiment of the invention the connecting means comprises a Y-connector.

The invention also provides an insufflator for insufflating a cavity in the body of a human or animal subject, the insufflator comprising a first control means for controlling the supply of insufflating gas to the cavity, a second control means for controlling application of a vacuum to the cavity, and a signal processor programmed to sequentially and alternately operate the first control means to supply insufflating gas to the cavity, and the second control means to apply a vacuum to the cavity in a plurality of sequential pressure/vacuum cycles for maintaining an intermittent flow of insufflating gas through the cavity to withdraw smoke and/or other undesirable gases from the cavity.

Preferably, the signal processor is programmed to operate the first control means to supply insufflating gas to the cavity for a predefined gas supply time period in each pressure/vacuum cycle.

Advantageously, the signal processor operates the second control means to apply vacuum to the cavity for a predefined vacuum application time period in each pressure/vacuum cycle.

Additionally, the invention provides a method for insufflating a cavity in the body of a human or animal subject with an insufflator operable in selectable first and second operating modes, the method comprising monitoring the pressure in the cavity (cavity pressure), operating the insufflator in the first operating mode for insufflating the cavity to maintain the cavity pressure at a set pressure, and in response to the cavity pressure exceeding the set pressure or a predefined upper pressure greater than the set pressure operating the insufflator to apply a vacuum to the cavity until the cavity pressure falls to the set pressure, or operating the insufflator to apply a vacuum to the cavity for a first predefined time period, and operating the insufflator in the second operating mode for sequentially and alternately supplying insufflating gas to the cavity and applying a vacuum to the cavity in a plurality of sequential pressure/vacuum cycles.

In one embodiment of the invention on failure of the cavity pressure to fall to or below the set pressure at the end of the first predefined time period, the insufflator is operated to apply the vacuum to the cavity for at least one second time period.

In another embodiment of the invention on failure of the cavity pressure to fall to or below the set pressure at the end of each second time period the insufflator is operated to apply the vacuum to the cavity for another second time period.

In another embodiment of the invention the time duration of the first one of the second time periods is computed as a function of the difference between the set pressure and the cavity pressure at the end of the first predefined time period.

In another embodiment of the invention the time duration of the first one of the second time periods is computed as a function of the rate at which the cavity pressure dropped during the first predefined time period.

In another embodiment of the invention the time duration of the second and each subsequent one of the second time periods is computed as a function of the difference between the set pressure and the cavity pressure at the end of the previous one of the second time periods.

In one embodiment of the invention the time duration of the second and each subsequent one of the second time periods is computed as a function of the rate at which the cavity pressure dropped during the previous one of the second time periods.

In one embodiment of the invention the application of the vacuum to the cavity is terminated at the end of each one of the second time periods, and the cavity pressure is monitored.

In another embodiment of the invention the application of the vacuum to the cavity is terminated at the end of the first predefined time period and the cavity pressure is monitored.

In another embodiment of the invention in the first operating mode on the cavity pressure exceeding the set pressure or the predefined upper pressure, the supply of insufflating gas to the cavity is terminated until the cavity pressure falls to or below the set pressure.

In one embodiment of the invention the second operating mode, the cavity pressure is maintained within a predefined pressure range about the set pressure during each pressure/vacuum cycle.

In one embodiment of the invention insufflating gas is supplied to the cavity during each pressure/vacuum cycle for a predefined gas supply time period.

In another embodiment of the invention vacuum is applied to the cavity during each pressure/vacuum cycle for a predefined vacuum application time period.

Preferably, the supply of insufflating gas to the cavity is interrupted during the predefined vacuum application time period of each pressure/vacuum cycle.

In another embodiment of the invention the application of vacuum to the cavity is interrupted during the predefined gas supply time period of each pressure/vacuum cycle,

In another embodiment of the invention the first operating mode the application of vacuum to the cavity is prevented when the first insufflating gas is being supplied to the cavity.

In another embodiment of the invention in the first operating mode the supply of insufflating gas to the cavity is prevented when vacuum is being supplied to the cavity.

In another embodiment of the invention in the first operating mode on the cavity pressure falling to or below the set pressure insufflating gas is supplied to the cavity for maintaining the cavity pressure at the set pressure.

In another embodiment of the invention application of vacuum to the cavity is terminated on the cavity pressure falling to our below a predefined lower pressure lower than the set pressure, and insufflating gas is supplied to the cavity until the cavity pressure returns to the set pressure.

In one embodiment of the invention insufflating gas is supplied to the cavity and the vacuum is applied to the cavity from the insufflator through a channel in an endoscope capable of accommodating insufflating gas to the cavity.

In one embodiment of the invention the said channel of the endoscope is connected to a first control means of the insufflator which controls the supply of insufflating gas to the cavity for accommodating insufflating gas to the cavity.

In another embodiment of the invention the said channel of the endoscope is connected to a second control means of the insufflator which controls the application of the vacuum to the cavity for applying the vacuum to the cavity.

In another embodiment of the invention the said channel of the endoscope is connected to the first and second control means through a connecting means.

Preferably, the cavity pressure is monitored through the said . channel of the endoscope. Alternatively, the cavity pressure is monitored through a channel of the endoscope other than the said channel.

In one embodiment of the invention the endoscope is inserted into the cavity through a bodily orifice of the subject.

Preferably, gases drawn from the cavity during application of vacuum to the cavity are filtered. Advantageously, the gases drawn from the cavity by the application of vacuum to the cavity are filtered through a bacterial-viral filter. Preferably, the filter is connected between the said channel of the endoscope and the insufflator.

In another embodiment of the invention water entrained in gases drawn from the cavity by the application of vacuum to the cavity is collected in a water collecting means. Preferably, the water collecting means is located between the said channel of the endoscope and the insufflator.

Further the invention provides a method for insufflating a cavity in the body of a human or animal subject comprising sequentially and alternately supplying insufflating gas to the cavity and applying a vacuum to the cavity in a plurality of sequential pressure/vacuum cycles for maintaining an intermittent flow of insufflating gas through the cavity to withdraw smoke and/or other undesirable gases from the cavity.

The invention further provides a method for insufflating a cavity in the body of a human or animal subject and for controlling the pressure at which the cavity is insufflated substantially at a set pressure, the method comprising inserting an endoscope into the cavity, connecting a channel of the endoscope capable of accommodating an insufflating gas to the cavity to an insufflator, and sequentially and alternately applying insufflating gas to the said channel of the endoscope for delivery to the cavity and applying a vacuum to the said channel of the endoscope in sequential pressure/vacuum cycles for withdrawing the smoke or the gas from the cavity with the cavity maintained substantially at the set pressure.

The advantages of the invention are many. By providing the insufflator as being operable in first and second operating modes, the insufflator is particularly suitable for insufflating a cavity during an endoscopic procedure during which smoke is generated, for example, by a low pressure procedure such as a low pressure dissecting procedure, and also in the same procedure during which high pressure gas, other than insufflating gas is produced in the cavity during the procedure, or is generated in the cavity during the procedure by, for example, an argon plasma coagulation procedure. By operating the insufflator in the first operating mode, which in general would be the normal operating mode of the insufflator, the cavity pressure is maintained at the set pressure, and in the event of high pressure gases being generated in the cavity during the endoscopic procedure or being introduced into the cavity during the endoscopic procedure, such gases are readily withdrawn to reduce the cavity pressure back to the set pressure by terminating the delivery of insuffiating gas to the cavity by the first control means, and operating the second control means to apply vacuum to the cavity for the first predefined time period, and if necessary, one or more second predefined time periods. Smoke generated in the cavity by, for example, a low pressure dissecting procedure, is readily removed by operating the insufflator in the second operating mode, whereby the cavity is subjected to the pressure/vacuum cycles, while at the same time maintaining the cavity pressure within plus or minus 1 mmHg or 2 mmHg about the set pressure.

By providing the insufflator with the pressure monitoring means selectively connected to the third port or the first or second outlet ports, the insufflator is suitable for use with an endoscope where only one channel is available for both insufflating the cavity and withdrawing gas from the cavity and also for monitoring the cavity pressure, and where a second channel is available in the endoscope, the cavity pressure may be monitored through the second channel by connecting the pressure monitoring means through the third port to the cavity, while insufflating of the cavity and withdrawing gases from the cavity are carried out through the first and second outlet ports, respectively, through the first channel of the cavity.

A further advantage of the invention is that by connecting the first control means and the second control means to the cavity through the instrument channel, due to the larger diameter of the instrument channel of an endoscope than the diameter of the insufflating channel thereof, significantly higher volumes of insufflating gas may be delivered to the cavity in the event of relatively high leakage from the cavity, and also relatively high volumes of gas may be withdrawn from the cavity in the event of the pressure exceeding the predefined upper pressure.

The invention will be more clearly understood from the following description of some preferred embodiments thereof which are given by way of example only with reference to the accompanying drawings, in which:

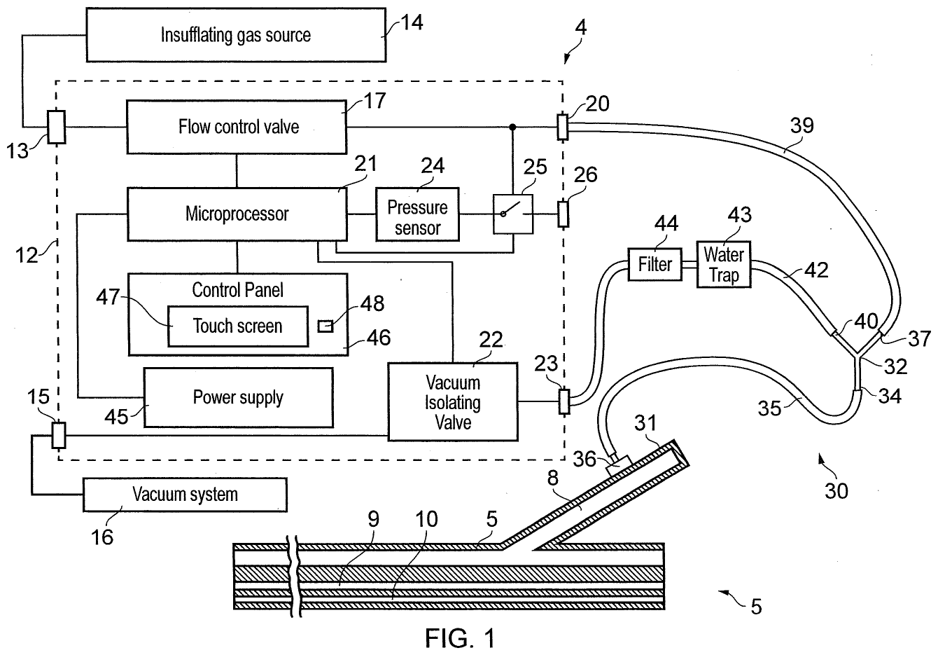

FIG. 1 is a block representation of an insufflating system according to the invention comprising an insufflator also according to the invention for insufflating a cavity in the body of a human or animal subject,

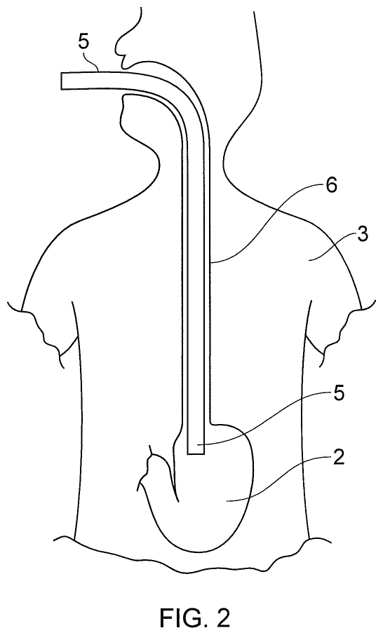

FIG. 2 is a front elevational view of a part of a human subject illustrating a part of the insufflating system extending into the stomach of the subject,

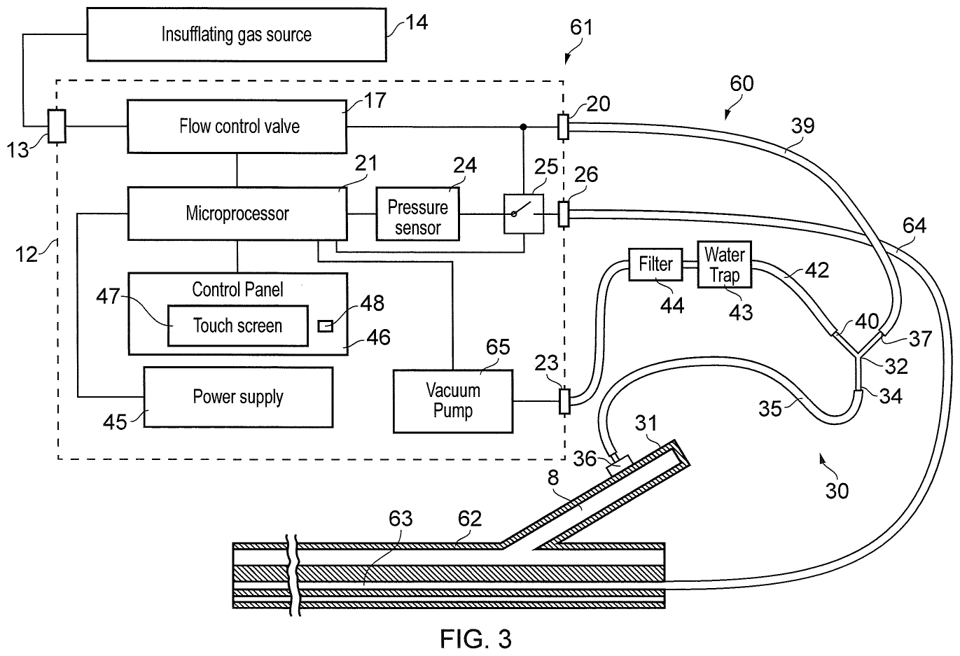

FIG. 3 is a block representation similar to that of FIG. 1 of an insufflating system according to another embodiment of the invention, and

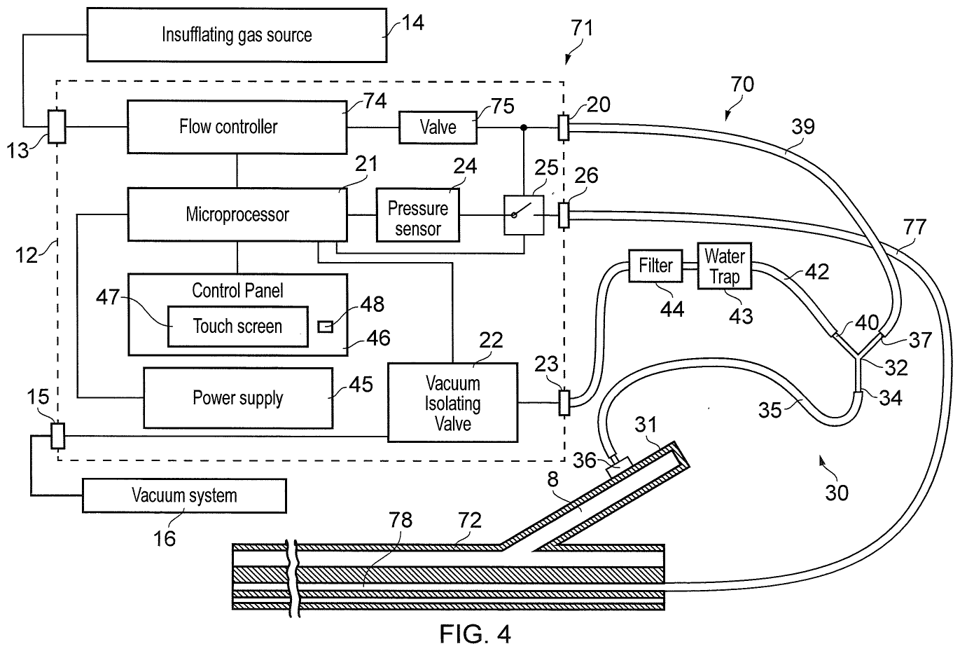

FIG. 4 is a block representation similar to the block representation of FIG. 1 of an insufflating system according to a further embodiment of the invention.

Referring to the drawings, and initially to FIGS. 1 and 2 thereof, there is illustrated an insufflating system according to the invention indicated generally by the reference numeral 1 for insufflating a cavity in the body of a human or animal subject. In this embodiment of the invention the insufflating system 1 will be described for insufflating a stomach 2 of a human subject 3.

The insufflating system 1 comprises an insufflator also according to the invention and indicated generally by the reference numeral 4 and an endoscope 5 connected to the insufflator 4. In this embodiment of the invention the endoscope 5 is entered orally through the oesophagus 6 into the stomach 2 of the subject 3. The endoscope 5 comprises a channel capable of supplying an insufflating gas to the stomach 2 of the subject 3, which in this embodiment of the invention comprises an instrument channel 8 of the endoscope 5, although, it will be appreciated that the channel capable of supplying insufflating gas to the stomach 2 may be any other suitable channel of the endoscope 5, for example, an insufflating channel 9 of the endoscope 5, a vacuum channel 10 of the endoscope 5 or may be a channel defined by an elongated tube releasably attached externally to the endoscope 5 and extending along the endoscope 5, which typically, would be used for insufflating the stomach 2. Such a tube having a channel extending therethrough for releasably attaching to an endoscope is disclosed in PCT Published Application Specification No. WO 2021/209983 of the present applicant.

The insufflator 4 as will be described in detail below is selectively and alternately operable in two operating modes, namely, a first operating mode and a second operating mode. In the first operating mode the insufflator 4 is configured to insufflate the stomach at a set pressure, and in the event of a high pressure gas, other than the insufflating gas, being generated in the stomach 2 or introduced into the stomach 2 during a procedure being carried out in the stomach 2, to automatically withdraw the high pressure gas, and any smoke which may be generated in the stomach 2 as a result of, for example, an argon plasma coagulation submucosal dissection procedure. In the second operating mode, the insufflator 4 is configured to insufflate the stomach 2 and to withdraw smoke generated in the stomach 2 during a low pressure dissection or coagulating procedure.

In the first operating mode the insufflator 4 insufflates the stomach 2 to maintain the pressure in the stomach 2, namely, the cavity pressure at the set pressure, which is selectable, and in this case is selectable in a range from 2 mmHg to 20 mmHg, and on the pressure in the stomach 2 exceeding a predefined upper pressure, for example, as a result of an argon plasma procedure, the insufflator automatically applies a vacuum to the stomach 2 to withdraw the high pressure gas and smoke, and to reduce the pressure in the stomach 2 to or below the set pressure. In this embodiment of the invention the predefined upper pressure is set at 1 mmHg above the set pressure, although the predefined upper pressure may be in the range of 0.5 mmHg to 5 mmHg above the set pressure. In the second operating mode, the insufflator 4 is configured to sequentially and alternately apply insufflating gas to the stomach 2 and apply a vacuum to the stomach 2 in continuous sequential pressure/vacuum cycles to maintain an intermittent flow of insufflating gas through the stomach 2 to withdraw smoke generated by the low pressure procedure, while at the same time maintaining the stomach 2 insufflated at the set pressure plus or minus 1 mmHg or 2 mmHg above the set pressure.

However, while the insufflating system 1 will be described for insufflating the stomach 2 in a human subject 3, it will be readily apparent to those skilled in the art that the insufflating system 1 may be used for insufflating any cavity in the body of a human or an animal subject.

Before describing the endoscope 5 and the connection of the insufflator 4 to the endoscope 5, the insufflator 4 will first be described.

The insufflator 4 comprises a housing 12. A first inlet port 13 is provided in the housing 12 for connecting the insufflator 4 to an external supply of pressurised insufflating gas, typically, a pressurised source of insufflating gas, and generally, a pressurised insufflating gas source 14 of carbon dioxide of the type commonly available in an operating theatre of a hospital. A second inlet port 15 is located in the housing 12 for connecting the insufflator 4 to a vacuum source, in this case a vacuum system 16 of the type commonly available in an operating theatre of a hospital.

A first control means for controlling the supply and the rate of the supply at which the insufflating gas is supplied from the insufflator 4 to the stomach 2 of the subject 3 comprises a flow control valve 17. The flow control valve 17 is located in the housing 12 and is connected between the first inlet port 13, and a first outlet port 20 located in the housing 12. The flow control valve 17 is operated under the control of a signal processor for controlling the supply and the rate of supply of the insufflating gas through the first outlet port 20 from the insufflator 4 to the stomach 2 for maintaining the pressure in the stomach 2 of the subject 3 at the set pressure. The signal processor, in this case comprises a microprocessor 21, although any other suitable signal processor may be used, for example, a microcontroller, a programmable logic controller or any other suitable signal processor.

A second control means for controlling application of a vacuum to the stomach 2 from the vacuum system 16 comprises an isolating valve 22 located in the housing 12 between the second inlet port 15, and a second outlet port 23 located in the housing 12. The isolating valve 22 is operable under the control of the microprocessor 21 between an isolating state with the vacuum system 16 isolated from the second outlet port 23, and a communicating state for selectively applying the vacuum from the vacuum system 16 to the second outlet port 23 and in turn to the stomach 2 of the subject 3, for in turn drawing smoke and argon gas from the stomach 2 and for reducing the pressure in the stomach 2 to the set pressure, as will be described in detail below.

A pressure monitoring means for monitoring pressure in the stomach 2 during insufflating thereof comprises a pressure sensor 24 located in the housing 12 for producing a signal indicative of the stomach pressure, which is read by the microprocessor 21. A solenoid operated two-way valve 25 operable under the control of the signal processor 21 selectively connects the pressure sensor 24 to the first outlet port 20 or to a third port 26, so that the stomach pressure may be monitored through either the first outlet port 20 or through the third port 26. The stomach pressure is monitored through the third port 26 if there is an additional available channel in the endoscope 5 other than the instrument channel 8 to allow the stomach pressure to be monitored continuously. In this embodiment of the invention neither the insufflating channel 9 or the vacuum channel 10 of the endoscope 5 are available to provide continuous monitoring of the stomach pressure, and the stomach pressure is monitored through the first outlet port 20.

The microprocessor 21 is programmed to read the signal produced by the pressure sensor 24 at predefined time intervals of approximately 1.5 seconds, and at the end of each predefined time interval, the microprocessor 21 operates the flow control valve 17 or the isolating valve 22 to pause the supply of insufflating gas to the stomach 2 or to pause the application of vacuum to the stomach 2, as the case may be, for a predefined monitoring time period of 0.5 seconds in order to allow the pressure at the pressure sensor 24 to equalise with the pressure in the stomach 2. At the end of each monitoring time period, the microprocessor 21 reads the signal from the pressure sensor 24, which is indicative of the stomach pressure. However, it is envisaged that the predefined intervals may be of time duration in the range of 1 second to 3 seconds, and the predefined monitoring time periods may be of time duration of 0.25 seconds to 0.75 seconds. On the other hand, if the third port 26 were connected directly through to the stomach 2 through another channel of the endoscope, the microprocessor 21 would be programmed to read the signal from the pressure sensor 24 at predefined time intervals of milliseconds duration, typically, of 10 milliseconds duration.

A connecting means comprising a tube set 30 connects the instrument channel 8 of the endoscope 5 adjacent a proximal end 31 thereof to the insufflator 4. The tube set 30 comprises a Y-connector 32 having a first port 34 connected by a first conduit 35 to the instrument channel 8 of the endoscope 5 through a port 36 of the endoscope 5 adjacent the proximal end 31 of the instrument channel 8. A second port 37 of the Y-connector 32 is connected through a second conduit 39 to the first outlet port 20 of the insufflator 4. A third port 40 of the Y-connector 32 is connected by a third conduit 42 to the second outlet port 23 of the insufflator 4 through a water collecting means, namely, a water trap 43 and a filter 44. Accordingly, the first outlet port 20 and the second outlet port 23 are connected through the tube set 30 to the instrument channel 8 of the endoscope 5 for sequentially and alternately supplying insufflating gas and applying a vacuum to the stomach 2 through the instrument channel 8 of the endoscope 5, as will be described below.

The filter 44 is provided for capturing bacteria, viruses, smoke and other matter drawn from the stomach 2, and in this embodiment of the invention comprises a bacterial-viral filter with a second activated carbon filter layer. The water trap 43 is provided for collecting water entrained in the gases drawn from the stomach 2, and protects the filter 44.

The microprocessor 21 as discussed above reads the signal from the pressure sensor 24 which is indicative of the stomach pressure, and in response to the read signal, the microprocessor 21 controls the operation of the flow control valve 17 for controlling the supply and the flow rate of insufflating gas to the stomach 2, and for selectively isolating the insufflating gas from the stomach 2. The microprocessor 21 also controls the isolating valve 22 in response to the signal read from the pressure sensor 24 for applying vacuum to the stomach 2, in the event of the stomach pressure exceeding the predefined upper pressure, as will be described below.

A power supply 45 located in the housing 12 powers the microprocessor 21 and the other components in the insufflator 4.

An interface, which may comprise a touch screen, a keypad, a voice recognition interface or any other suitable interface, is located in a control panel 46 in the housing 12 for inputting data, to the microprocessor 21, namely, the selected set pressure at which the stomach 2 is to be maintained during insufflating thereof, and other data necessary for the operation of the insufflator 4. In this case, the interface comprises a touch screen 47. The touch screen 47 also displays data relating to the insufflating of the stomach 2, such as the stomach pressure and other relevant data. A membrane pushbutton operated switch 48 is also located in the control panel 46 in the housing 12 for selecting and switching between the two operating modes of the insufflator 4.

The microprocessor 21 is programmed to operate the insufflator 4 in the two operating modes. In the first operating mode of the insufflator 4, the microprocessor 21 is programmed to operate the flow control valve 17 for supplying insufflating gas to the stomach 2 in order to maintain the stomach pressure at the set pressure. Initially on commencement of insufflating of the stomach 2, the microprocessor 21 operates the isolating valve 22 into the isolating state to isolate the second outlet port 23 from the vacuum system 16, and operates the flow control valve 17 to supply insufflating gas through the first outlet port 20, and in turn through the instrument channel 8 of the endoscope 5 to the stomach 2 for insufflating thereof. At the predefined time intervals of 1.5 seconds, the microprocessor 21 operates the flow control valve 17 to pause insufflating of the stomach 2 for the predefined monitoring time period of 0.5 seconds for monitoring the pressure in the stomach 2. At the end of each predefined monitoring time period, the microprocessor 21 reads the signal indicative of the stomach pressure from the pressure sensor 24. On the signal read from the pressure sensor 24 being indicative of the stomach pressure being below the set pressure, the microprocessor 21 is programmed to operate the flow control valve 17 to increase the flow rate of insufflating gas to the stomach 2 in order to return the stomach 2 to the set pressure and to maintain the stomach pressure at the set pressure.

If however, the signal read from the pressure sensor 24 is indicative of the stomach pressure having exceeded the predefined upper pressure, for example, as a result of an argon plasma coagulation being employed in the procedure being carried out in the stomach 2, the microprocessor 21 is programmed to operate the flow control valve 17 to isolate the insufflating gas from the first outlet port 20 and to simultaneously operate the isolating valve 22 from the isolating state to the communicating state for applying vacuum to the second outlet port 23 for a first predefined time period of approximately 3 seconds, for in turn applying vacuum to the stomach 2 in order to draw the high pressure argon gas from the stomach 2 and to reduce the stomach pressure to the set pressure. At the end of the first predefined time period, the microprocessor 21 operates the isolating valve 22 into the isolating state and reads the signal from the pressure sensor 24.

If the signal read from the pressure sensor 24 at the end of the first predefined time period is indicative of the stomach pressure having fallen to or below the set pressure, the microprocessor 21 operates the flow control valve 17 to reinstate the supply of insufflating gas to the stomach 2 to maintain the pressure in the stomach 2 at the preset pressure as already described, and leaves the isolating valve 22 in the isolating state.

However, if the signal read from the pressure sensor 24 at the end of the first predefined time period is not indicative of the stomach pressure having fallen to or below the set pressure, the microprocessor 21 is programmed to operate the isolating valve 22 again into the communicating state for at least one second time period to again apply the vacuum to the stomach 2. At the end of the first one of the second time periods, the microprocessor 21 operates the isolating valve 22 into the isolating state and reads the signal from the pressure sensor 24. If at the end of the first one of the second time periods the stomach pressure has fallen to or below the set pressure, the microprocessor 21 is programmed to operate the flow control valve 17 to reinstate the supply of insufflating gas to the stomach 2 to maintain the stomach pressure at the preset pressure as already described, and the microprocessor 21 leaves the isolating valve 22 in the isolating state.

However, if the signal read from the pressure sensor 24 at the end of the first one of the second time periods is not indicative of the stomach pressure having fallen to or below the set pressure, the signal processor 21 is programmed to again operate the isolating valve 22 into the communicating state to again apply the vacuum to the second outlet port 23, and in turn to the stomach 2 for another one of the second time periods, and so on until the pressure in the stomach 2 has fallen to or below the set pressure.

In this embodiment of the invention the first predefined time period and the second time period are of the same time duration, and are each of approximately 3 seconds, but may lie in the range of 1 second to 5 seconds. In other embodiments of the invention the second time periods may be of shorter time duration than the time duration of the first predefined time period, and in which case the time duration of the second time periods may be the same or the time durations thereof may be different, and in general, would decrease progressively.

In another embodiment of the invention the time duration of the first one of the second time periods is computed by the microprocessor 21 as a function of the difference between the set pressure and the stomach pressure read at the end of the first predefined time period, and the rate at which the stomach pressure dropped during the first predefined time period, so that at the end of the first one of the second time periods, the stomach pressure should fall to the set pressure. The duration of each one of the second and subsequent ones of second time periods is computed by the microprocessor 21 as a function of the difference between the set pressure and the stomach pressure at the end of the immediately preceding second time period, and the rate at which the stomach pressure fell during the immediately preceding second time period, so that at the end of the second time period just to be commenced, the stomach pressure should fall to the set pressure.

On the other hand, if an additional channel were available in the endoscope 5 for monitoring pressure in the stomach 2, the third port 26 would be connected through a conduit to that additional channel of the endoscope, and the microprocessor 21 would read the signal indicative of the pressure in the stomach 2 from the pressure sensor 24 at predefined time intervals, typically, of 10 milliseconds. Otherwise, operation of the insufflator 4 would be similar to that just described.

In the second operating mode of the insufflator 4, the microprocessor 21 is programmed to operate the insufflator 4 for sequentially and alternately supplying insufflating gas and applying a vacuum to the stomach 2 in sequential pressure/vacuum cycles of duration of approximately 10 seconds per cycle, in order to provide an intermittent flow of insufflating gas through the stomach 2, to draw smoke and low pressure gases introduced or generated in the stomach 2 from the stomach 2. During each pressure/vacuum cycle, the microprocessor 21 is programmed to operate the flow control valve 17 to supply insufflating gas to the stomach 2 for a predefined gas supply time period, and at the end of the predefined gas supply time period, to operate the isolating valve 22 into the communicating state for a predefined vacuum application time period to apply vacuum to the second outlet port 23, and in turn to the stomach 2. Each predefined gas supply time period is of approximately 7 seconds, and each predefined vacuum application time period is of approximately 3 seconds. During each predefined gas supply time period, the isolating valve 22 is operated in the isolating state to prevent vacuum being applied to the stomach 2, and during each predefined vacuum application time period, the flow control valve 17 is operated to isolate the first outlet port 20 from the insufflating gas source 14 to prevent insufflating gas being delivered to the stomach 2.

The alternating of the supply of insufflating gas and the application of the vacuum to the stomach 2 during each pressure/vacuum cycle maintains a flow of insufflating gas through the stomach to remove any smoke generated in the stomach 2 during, for example, a low pressure dissection procedure being carried out therein, and also to remove any low pressure gases introduced to or generated in the stomach 2 during the procedure, while at the same time the pressure in the stomach is maintained at the set pressure plus or minus 1 mmHg or 2 mmHg about the set pressure.

In use, the insufflator 4 is connected to the insufflating gas source 14 and to the vacuum system 16. The endoscope 5 is inserted orally through the oesophagus 6 into the stomach 2 of the subject 3, and the insufflator 4 is connected to the endoscope 5. The first outlet port 20 and the second outlet port 23 are connected to the instrument channel 8 of the endoscope 5 through the tube set 30. The set pressure at which the stomach 2 is to be insufflated is entered into the microprocessor 21 through the touch screen 47. In the absence of a conduit being connected to the third port 26 for connecting the third port 26 to a channel in the endoscope 5, other than the instrument channel 8, the microprocessor 21 operates the valve 25 to connect the pressure sensor 24 to the first outlet port 20. Alternatively, if the two-way valve 25 instead of being provided as a solenoid operated valve operated under the control of the microprocessor, were provided as a manually operated valve, the two-way valve would be manually operated to connect the pressure sensor 24 to the first outlet port 20.

The operating mode in which the insufflator 4 is to be operated is selected by operating the membrane button operated switch 48 in the control panel 46 in order to select the operating mode in which the insufflator 4 is to operate. If an operating mode is not selected, the insufflator 4 defaults to the first operating mode. In general, it is envisaged that the first operating mode will normally be selected to commence insufflating of the stomach 2. Once the first operating mode is selected, or in default, the microprocessor 21 is programmed to operate the insufflator 4 in the first operating mode as already described and, the microprocessor 21 continues to operate the insufflator 4 in the first operating mode during the carrying out of the procedure in the stomach 2, or until the second operating mode is selected by operating the membrane button operated switch 48. On the second operating mode being selected, the microprocessor 21 is programmed to operate the insufflator 4 in the second operating mode as already described until the procedure has been completed, or until the first operating mode is again selected by operating the membrane button operated switch 48.

If the insufflator 4 is to be operated initially in the second operating mode, the second operating mode is selected by operating the membrane button operated switch 48, and the insufflator 4 continues to operate in the second operating mode until the procedure has been completed or the first operating mode is selected by operating the membrane button operated switch 48.

In general, it is envisaged that the insufflator 4 will be operated in the first operating mode in procedures where a high pressure gas, other than the insufflating gas, is likely to be introduced into the stomach 2 being insufflated or is likely to be generated in the stomach 2 during the carrying out of the procedure and also where smoke is likely to be generated in the cavity during a high pressure dissection or coagulation process. The insufflator 4, in general, will also be operated in the first mode of operation where it is unlikely that a high pressure gas, other than the insufflating gas, will be introduced to the stomach 2 or generated therein during the procedure. However, the second operating mode of the insufflator 4 will be selected prior to or just prior to the commencement of a procedure during which smoke will be generated in, for example, a low pressure dissecting or coagulation procedure for withdrawing the smoke while maintaining the stomach pressure at the set pressure plus or minus 1 mmHg or 2 mmHg about the set pressure.

Referring now to FIG. 3 there is illustrated an insufflating system according to another embodiment of the invention indicated generally by the reference numeral 60. The insufflating system 60 is substantially similar to the insufflating system 1 and similar components are identified by the same reference numerals. The insufflating system 60 comprises an insufflator also according to the invention and indicated generally by the reference numeral 61 and an endoscope 62 connected to the insufflator 61 for supplying insufflating gas to a cavity to be insufflated in the body of a human or animal subject and for applying a vacuum thereto. The endoscope 62 is similar to the endoscope 5 of the insufflating system 1, and the instrument channel 8 thereof is connected to the insufflator 61 by a tube set 30, similar to the tube set 30 of the insufflating system 1, and is connected to the insufflator 61 by the tube set 30 in a similar manner as the endoscope 5 is connected to the insufflator 4 of the insufflating system 1. The only difference between the insufflating system 60 and the insufflating system 1 lies firstly in the insufflator 61, and secondly, in the manner in which the cavity pressure is monitored by the pressure sensor 24.

Dealing initially with the monitoring of the cavity pressure, in this case, due to the procedure being carried out in the cavity, an additional channel of the endoscope 62 is available for monitoring pressure in the cavity, namely, the channel 63 of the endoscope 62, which in this case is an insufflating channel 63 of the endoscope 62. Accordingly, the third port 26 is connected to the channel 63 of the endoscope 62 through a conduit 64, so that the pressure sensor 24 may continuously monitor the cavity pressure, and the microprocessor 21 is programmed to monitor the cavity pressure at millisecond intervals, typically at 10 millisecond intervals.

Turning now to the insufflator 61, in this embodiment of the invention the second control means of the insufflator 61 comprises a vacuum generating means, in this embodiment of the invention a vacuum pump 65 located in the housing 12 of the insufflator 61 and connected directly to the second outlet port 23. The vacuum pump 65 is operated under the control of the microprocessor 21 in response to the signal read from the pressure sensor 24 in a similar manner as the isolating valve 22 of the insufflator 4 is operated for applying vacuum to the second outlet port 23. Since the insufflator 61 comprises the vacuum pump 64, the second inlet port 15 may be dispensed with.

Accordingly, in this embodiment of the invention the microprocessor 21 when operating the insufflator 61 in the first and second operating modes, when a vacuum is to be applied to the cavity being insufflated, the microprocessor 21 operates the flow control valve 17 to isolate the first outlet port 20 from the insufflating source 14, as already described with reference to the insufflator 4, and operates the vacuum pump 65 to apply a vacuum to the second outlet port 23 and in turn to the cavity, instead of operating the isolating valve 22 from the isolating state to the communicating state.

However, since the pressure in the cavity is monitored continuously by the pressure sensor 24, and the microprocessor 21 monitors the cavity pressure at the 10 millisecond intervals, when operating in the first operating mode when the pressure in the cavity exceeds the predefined upper pressure, instead of operating the vacuum pump 65 for applying a vacuum to the cavity for a first predefined time period, once the vacuum pump has been operated to apply the vacuum to the cavity and the flow control valve 17 has been operated to isolate the cavity from the insufflating gas source 14, the microprocessor 21 reads the signal from the pressure sensor 24 at the 10 millisecond intervals, and on the signal read from the pressure sensor 24 being indicative of the cavity pressure having fallen to or below the set pressure, the microprocessor 21 deactivates the vacuum pump 65 and simultaneously operates the flow control valve 17 to reinstate insufflating gas to the cavity for maintaining the cavity at the set pressure.

Otherwise, the insufflating system 60 and its operation is similar to that of the insufflating system 1.

Referring now to FIG. 4, there is illustrated an insufflating system according to another embodiment of the invention indicated generally by the reference numeral 70 for insufflating a cavity in the body of a human or animal subject. The cavity may be the stomach of a subject, or any other cavity. The insufflator system 70 comprises an insufflator 71 and an endoscope 72. The insufflator 71 is substantially similar to the insufflator 4 described with reference to FIGS. 1 and 2, and similar components are identified by the same reference numerals. The insufflator 71 is operable in two operating modes, namely, a first operating mode and a second operating mode substantially similar to the two operating modes of the insufflator 4 of the insufflating system of FIGS. 1 and 2. The endoscope 72 is also substantially similar to the endoscope 5 of the insufflating system 1, described with reference to FIGS. 1 and 2, and similar components are also identified by the same reference numerals.

In the insufflator 71, instead of providing the first control means as a flow control valve, the first control means in the insufflator 71 comprises a flow controller 74 and a separate insufflating gas isolating valve 75. The flow controller 74 controls the flow rate of insufflating gas from the insufflating gas source 14 to the first outlet port 20, and in turn to the cavity being insufflated. The insufflating gas isolating valve 75 comprises a solenoid operated isolating valve selectively and alternately operable in an isolating state isolating the flow controller 74 from the first outlet port 20, and a communicating state communicating the flow controller 74 with the first outlet port 20 for accommodating insufflating gas from the flow controller 74 to the first outlet port 20.

The flow controller 74 is operated under the control of the microprocessor 21 in a substantially similar manner as the flow control valve 17 of the insufflator 4 is operated for controlling the flow rate of insufflating gas to the cavity. The insufflating gas isolating valve 75 is operated under the control of the microprocessor 21 between the isolating state to prevent the flow of insufflating gas from the flow controller to the first outlet port 20 and the communicating state to permit the supply of insufflating gas from the insufflating gas source 14 to the outlet port 20 in a substantially similar manner as the microprocessor 21 operates the flow control valve 17 to control the supply of insufflating gas from the insufflating gas source 14 to the outlet port 20 and in turn to the cavity being insufflated.

Accordingly, when insufflating gas is to be delivered to the cavity, the microprocessor 21 operates the insufflating gas isolating valve 75 into the communicating state, and when insufflating gas from the insufflating gas source 14 is to be isolated from the cavity being insufflated, the microprocessor 21 operates the insufflating gas isolating valve 75 into the isolating state to isolate the first outlet port 20 and in turn the cavity from the insufflating gas source 14.

Additionally, in this embodiment of the invention the pressure sensor 24 is connected to the third port 26 only, and cavity pressure is monitored by the pressure sensor 24 through the third port 26 and in turn through a conduit 77 connecting the second port 26 to one of the channels of the endoscope 72, other than the said instrument channel 8 thereof. For example, the pressure sensor 24 may be connected through the conduit 77 to an insufflating channel 78 or a vacuum channel if such were available in the endoscope 72.

In this embodiment of the invention the first outlet port 20 and the second outlet port 23 of the insufflator 71 are connected to the instrument channel 8 of the endoscope 72 adjacent the proximal end 31 of the endoscope 72 through the port 36 by a tube set 30 similar to the tube set 30 of the insufflating system described with reference to FIGS. 1 and 2 which connects the insufflator 4 to the endoscope 5 of the insufflating system 1, and in a similar manner as the tube set 30 connects the insufflator 4 to the endoscope 5.

The operation of the insufflating system 70, in general, is substantially similar to that of the operation of the insufflating system 1. However, in this embodiment of the invention since the pressure sensor 24 is continuously connected to the cavity being insufflated through the conduit 77 and the insufflating channel 78 of the endoscope 72, the microprocessor 21 is programmed to read the signal produced by the pressure sensor 24 indicative of the cavity pressure at the predefined time intervals of 10 milliseconds, and therefore, the operation of the insufflator 71 in the first and operating mode is similar to the operation of the insufflator 61 described with reference to FIG. 3 in the first operating mode. Operation of the insufflator 71 in the second operating mode is similar to the operation of the insufflator 4 described with reference to FIGS. 1 and 2 in the second operating mode.

Additionally, in this embodiment of the invention, if at any time while the vacuum isolating valve 22 is being operated in the communicating state for applying vacuum to the cavity, in either the first or second operating modes, the signal read from the pressure sensor 24 by the microprocessor 21 is indicative of the cavity pressure falling below a predefined lower pressure, the microprocessor 21 is programmed to operate the vacuum isolating valve 22 from the communicating state to the isolating state, and to simultaneously operate the insufflating gas isolating valve 75 from the isolating state to the communicating state and to operate the flow controller 74 to supply the insufflating gas at the flow rate at which insufflating gas had been delivered to the cavity prior to the insufflating gas isolating valve 75 being operated into the isolating state, or to increase the flow rate of the insufflating gas above that flow rate, in order to supply insufflating gas to the cavity to increase the cavity pressure to the set pressure.

Once the signal read from the pressure sensor 24 by the microprocessor 21 is indicative of the cavity pressure having returned to the set pressure, the microprocessor 21 operates the flow controller to reduce the flow rate of insufflating gas to the cavity in order to maintain the cavity pressure at the set pressure. However, if the cavity pressure continues to rise above the set pressure and reaches the predefined upper pressure, the microprocessor 21 is programmed to operate the insufflating gas isolating valve 75 from the communicating state to the isolating state and simultaneously to operate the vacuum isolating valve 22 from the isolating state to the communicating state until the cavity pressure has fallen to the set pressure. In this embodiment of the invention the predefined lower pressure is approximately 6 mmHg below the set pressure. Although in some embodiments of the invention it is envisaged that the predefined lower pressure may lie in the range of 2 mmHg to 8 mmHg below the set pressure, while in other embodiments of the invention the predefined lower pressure may lie in the range of 2 mmHg to 10 mmHg below the set pressure.

Otherwise, the insufflator 71 and the insufflating system 70 and their operation are similar to that of the insufflator 4 and the insufflating system 1 described with reference to FIGS. 1 and 2.

While the predefined upper pressure has been described as being 1 mmHg above the set pressure, it is envisaged that the predefined upper pressure may be any upper safe pressure to which the cavity may be insufflated, and will largely be dependent on the nature of the cavity being insufflated. However, typically, in the case of a stomach, it is envisaged that the predefined upper pressure will lie in the range of 0.5 mmHg to 5 mmHg above the set pressure, and preferably, will lie in the range of 0.5 mmHg to 4 mmHg above the set pressure, provided of course that the set pressure is set at a pressure such that the difference between the set pressure and the predefined upper pressure does not result in the predefined upper pressure being of a dangerously high pressure value.

While the insufflators 4, 61 and 71 have been described when operating in the second mode to maintain the pressure in the cavity at the set pressure plus or minus 1 mmHg or 2 mmHg, in some embodiments of the invention it is envisaged that the pressure range within which the cavity pressure would be maintained during insufflating thereof in the second operating mode could be maintained at the set pressure plus or minus up to 3 mmHg.

While the means for switching the operation of the insufflators 4, 61 and 71 between the first and second operating modes has been described as comprising a membrane button operated switch, any other suitable means for switching the insufflators between the first and second operating modes may be provided. In some embodiments of the invention it is envisaged that the means for switching the insufflators between the first and second operating modes may comprise a foot pedal operated switch, which would be operated typically, by the surgeon or clinician. The foot pedal operated switch would be connected into the insufflator to the microprocessor.

While the insufflators 4, 61 and 71 have been described as forming a part of an insufflating system, the insufflators 4, 61 and 71, as well as any other insufflators according to the invention may be operated as standalone insufflators, and in which case, the insufflators may be connected to the cavity being insufflated by any suitable means, for example, through a colonoscope or a trocar, depending on the manner in which the procedure is being carried out.

While the first control means has been described as comprising a flow control valve, in some embodiments of the invention it is envisaged that the first control means may comprise a flow controller, which would not necessarily act to isolate the first outlet port from the insufflating gas source, and in which case, it is envisaged that as well as the flow controller, an isolating valve would be located between the flow controller and either the first outlet port or the first inlet port, and more commonly, would be located between the flow controller and the first outlet port.

While the insufflating systems 1, 60 and 70 have been described as comprising a bacterial-viral filter, any other suitable filter may be used, and while the filter has been described as being located in the third conduit, the filter may be located in any suitable location from the third outlet port of the Y-connector to and including the second inlet port 15. In some embodiments of the invention the filter may be located in the housing between the isolating valve or the vacuum pump and the second inlet or outlet port.

While the insufflating systems 1, 60 and 70 have been described as comprising a water collecting means, in some embodiments of the invention the water collecting means may be omitted. It is also envisaged that other suitable water collecting means may be provided, besides a water trap. Additionally, where a water collecting means is provided, it will normally be located upstream of the filter in order to protect the filter from water entrained in the gases being evacuated from the cavity.

While the signal processor has been described as comprising a microprocessor, any other suitable signal processor may be used, for example, a microcontroller, a programmable logic controller or indeed any other suitable signal processor.

It will also of course be appreciated that any other suitable connecting means for connecting the insufflator to the endoscope may be used besides the tube set 30.

While the insufflating systems 1, 60 and 70 according to the invention and the insufflators according to the invention have been described for use in the carrying out of a procedure in the stomach of a subject, it will be readily apparent to those skilled in the art that the insufflating systems and the insufflators according to the invention may be used for carrying out any other procedures, for example, a procedure in the rectum, the colon, the intestine, the oesophagus, the abdominal cavity and in carrying out a procedure in the rectum or the colon where a colonoscope is being used, the colonoscope would be entered rectally into the subject. When the procedure is being carried out in the intestine of a subject, the entry of the endoscope or colonoscope into the subject would depend on the location in the intestine in which the procedure is being carried out. In the case of a procedure being carried out in the intestine closer to the stomach than the colon, it is envisaged that the endoscope would be entered orally into the subject, and otherwise, the colonoscope would be entered rectally into the subject.

While the set pressure has been described as being selectable in the range of 2 mmHg to 20 mmHg, it is envisaged that in some embodiments of the invention the set pressure may be selectable up to 25 mmHg. It will also be appreciated that while the set pressure has been described as being selectable, in some embodiments of the invention it is envisaged that the set pressure may be preset between 1 mmHg and 20 mmHg, and in some cases up to 25 mmHg.

While the second control means in the insufflators 4 and 71 have been described as comprising an isolating valve which is operable in two states only, namely, in an isolating state and a communicating state for controlling the supply of vacuum, it is envisaged that in some embodiments of the invention the second control means may comprise a vacuum pressure control valve, which would allow the pressure of the vacuum applied to the second outlet port to be varied and selectable. In which case, the microprocessor or other signal processor would be configured to control the vacuum pressure control valve in response to a signal from a switch means, for example, an external foot pedal operated switch configured to produce a signal to the microprocessor or other signal processor indicative of the vacuum pressure to be applied to the cavity. Typically, such a foot pedal or indeed a manually operated switch, may comprise a rheostat, so that the further the foot pedal is depressed, the greater would be the vacuum applied to the cavity, and in the case of a manually operated switch, for example, a button operated switch, the more the button operating the switch is depressed, the greater would be the vacuum applied to the cavity.

It is also envisaged that the microprocessor or other suitable signal processor may be configured to control the rate at which the vacuum pump applies the vacuum to the cavity, for in turn varying the vacuum applied to the cavity. In which case, it is envisaged that the microprocessor or signal processor would be configured to be responsive to a signal from a suitable switch means, for example, a foot pedal operated switch or a manually operated button switch, such as a rheostat switch, so that the more the pedal is depressed or the button of the button operated switch is depressed, the greater would be the vacuum applied to the cavity by the vacuum pump.

While in the embodiments of the invention described with reference to FIGS. 1 to 3, a valve 25 has been provided operable under the control of the microprocessor 21 for selectively switching the pressure sensor 24 between the third port 26 and the first outlet port 20, in some embodiments of the invention it is envisaged that the valve 25 may be omitted, and in which case, two pressure sensors may be provided, one pressure sensor being connected to the third port 26 and the other pressure sensor being connected to the first outlet port 20. The microprocessor 21 would be programmed to read the signals from the appropriate one of the pressure sensors. If the third port 26 is not connected to the cavity being insufflated through the endoscope or otherwise, the microprocessor would read the signal from the pressure sensor connected to the first outlet port 20, and if the third port 26 is connected for communicating with the cavity being insufflated, the microprocessor would read the signal from the pressure sensor connected to the third port 26. It is also envisaged that the pressure sensor may be connected to the second outlet port 23 and could also monitor the cavity pressure through the second outlet port.

It is also envisaged that three separate pressure sensors may be provided, one of which would communicate with the first outlet port 20, the other of which would communicate with the second outlet port 23, and the third of which would communicate with the third port 26, and the microprocessor would be configured to read the signal from the appropriate one of the three pressure sensors to determine the cavity pressure.

It will also be appreciated that a switch other than a membrane switch may be provided for selecting the operating mode of the insufflators described with reference to FIGS. 1 to 4.

It is also envisaged that in some embodiments of the invention a means for selectively connecting the tube set between the instrument channel and a channel other than the instrument channel, for example, an insufflating channel or a vacuum channel of the endoscope may be provided. In which case, it is envisaged that the tube set would be connected through a spool valve or other suitable valve to both the instrument channel and the other one of the channels of the endoscope, and the spool valve or such other suitable valve would typically be operated by a foot pedal operated switch which would allow the tube set to be selectively and alternately connected to the instrument channel or the other one of the channels of the endoscope, such as the insufflating channel or the vacuum channel as the case may be.

1-130. (canceled)

131. An insufflator for insufflating a cavity in the body of a human or animal subject, the insufflator comprising a first control means for controlling the supply of insufflating gas to the cavity, a second control means for controlling application of a vacuum to the cavity, a pressure monitoring means for monitoring pressure in the cavity (cavity pressure) and for producing a signal indicative of the cavity pressure, and a signal processor configured to read the signal from the pressure monitoring means and to control the first and second controls means in response to the signal read from the pressure monitoring means, wherein the signal processor is configured to selectively operate the insufflator in selectable ones of a first operating mode and a second operating mode, and in the first operating mode the signal processor is configured to operate the first control means in response to the signal read from the pressure monitoring means to control the supply of insufflating gas to the cavity for maintaining the cavity pressure substantially at a set pressure, and on the signal read from the pressure monitoring means being indicative of the cavity pressure exceeding a predefined upper pressure greater than the set pressure, the signal processor is configured to operate the second control means to apply a vacuum to the cavity until the cavity pressure falls to the set pressure, or to apply a vacuum to the cavity for a first predefined time period, and in the second operating mode, the signal processor is configured to control the first and second control means to sequentially and alternately supply insufflating gas to the cavity and to apply a vacuum to the cavity in a plurality of sequential pressure/vacuum cycles.

132. An insufflator as claimed in claim 131 in which the first predefined time period lies in the range of 1 second to 5 seconds, and preferably, the first predefined time period is approximately 3 seconds, and advantageously, the signal processor is responsive to failure of the cavity pressure falling to or below the set pressure at the end of the first predefined time period to operate the second control means to apply the vacuum to the cavity for at least one second time period, and preferably, the signal processor is responsive to failure of the cavity pressure falling to or below the set pressure at the end of each second time period to operate the second control means to apply the vacuum to the cavity for another second time period, and advantageously, the signal processor is programmed to compute the time duration of the first one of the second time periods as a function of the difference between the set pressure and the cavity pressure at the end of the first predefined time period, and preferably, the signal processor is programmed to compute the time duration of the first one of the second time periods as a function of the rate at which the cavity pressure dropped during the first predefined time period, and advantageously, the signal processor is programmed to compute the time duration of the second and each subsequent one of the second time periods as a function of the difference between the set pressure and the cavity pressure at the end of the previous one of the second time periods, and preferably, the signal processor is programmed to compute the time duration of the second one and each subsequent one of the second time periods as a function of the rate at which the cavity pressure dropped during the previous one of the second time periods, and advantageously, the time duration of each second time period is shorter than the time duration of the first predefined time period, and preferably, the time duration of each subsequent second time period is shorter than the time duration of the immediately previous second time period, alternatively, the time duration of each one of the second time periods is the same as the time duration of the first predefined time period.

133. An insufflator as claimed in claim 132 in which the signal processor is programmed to operate the second control means to terminate the application of the vacuum to the cavity at the end of each one of the second time periods, and to read the signal from the pressure monitoring means, and preferably, the signal processor is programmed to operate the second control means to terminate the application of the vacuum to the cavity at the end of the first predefined time period and to read the signal from the pressure monitoring means, and advantageously, in the first operating mode of the insufflator the signal processor is responsive to the cavity pressure exceeding the predefined upper pressure to operate the first control means to terminate the supply of insufflating gas to the cavity until the cavity pressure falls to or below the set pressure, and preferably, the predefined upper pressure lies in the range of 0.5 mmHg to 5 mmHg above the set pressure.

134. An insufflator as claimed in claim 131 in which in the second operating mode, the signal processor is programmed to operate the first and second control means during each pressure/vacuum cycle to maintain the cavity pressure within a predefined pressure range about the set pressure, and preferably, the predefined pressure range about the set pressure lies in the range of 3 mmHg above the set pressure and 3 mmHg below the set pressure, and advantageously, the second operating mode, the signal processor is programmed to operate the first control means to supply the insufflating gas to the cavity for a predefined gas supply time period in each pressure/vacuum cycle, and preferably, in the second operating mode, the signal processor is programmed to operate the second control means to apply vacuum to the cavity for a predefined vacuum application time period in each pressure/vacuum cycle, and advantageously, each predefined vacuum application time period lies in the range of 1 second to 5 seconds, and preferably, the duration of the predefined vacuum application time period lies in the range of one eighth to one half of the duration of each pressure/vacuum cycle, and advantageously, the duration of the predefined vacuum application time period is approximately one third of the duration of each pressure/vacuum cycle, and preferably, the signal processor is programmed to operate the first control means to prevent insufflating gas being supplied to the cavity during the predefined vacuum application time period of each pressure/vacuum cycle, and advantageously, each predefined gas supply time period lies in the range of 4 seconds to 10 seconds, and preferably, in the second operating mode the signal processor is programmed to operate the second control means to prevent vacuum being applied to the cavity during the predefined gas supply time period of each pressure/vacuum cycle.

135. An insufflator as claimed in claim 131 in which in the first operating mode the signal processor is programmed to operate the second control means to prevent vacuum being applied to the cavity when the first control means is being operated to supply insufflating gas to the cavity, and preferably, in the first operating mode the signal processor is programmed to operate the first control means to prevent insufflating gas being supplied to the cavity when the second control means is being operated to apply a vacuum to the cavity, and advantageously, in the first operating mode the signal processor is responsive to the signal from the pressure monitoring means being indicative of the cavity pressure falling to or below the set pressure to operate the first control means to supply insufflating gas to the cavity for maintaining the cavity pressure at the set pressure, and preferably, the signal processor is programmed to be responsive to the signal read from the pressure sensing means being indicative of the cavity pressure having fallen to a predefined lower pressure lower than the set pressure during operation of the second control means applying vacuum to the cavity, to operate the second control means to terminate application of vacuum to the cavity, and to operate the first control means to supply insufflating gas to the cavity until the cavity pressure returns to the set pressure, and preferably, the predefined lower pressure lies in the range of 2 mmHg to 10 mmHg below the set pressure, and advantageously, the set pressure lies in the range of 2 mmHg to 20 mmHg, and preferably, is selectable.

136. An insufflator as claimed in claim 131 in which the insufflator comprises a first outlet port communicating with the first control means for accommodating insufflating gas therethrough to the cavity, and preferably, the insufflator comprises a second outlet port communicating with the second control means through which vacuum is applied to the cavity, and advantageously, the pressure monitoring means is connected to the first outlet port or to the second outlet port, or to both the first and second outlet ports, and preferably, the insufflator comprises a third port, and the pressure monitoring means is connected to or is selectively connectable to the third port or to one or both of the first or second outlet ports, and preferably, the insufflator comprises an insufflating gas source communicating with the first control means, or alternatively, the insufflator comprises a first inlet port communicating with the first control means for receiving insufflating gas from an external insufflating gas source thereof, and preferably, the first control means comprises a flow control valve, and preferably, the second control means comprises a vacuum pump, or alternatively, the insufflator comprises a second inlet port communicating with the second control means for coupling the insufflator to an external vacuum source, and preferably, the second control means comprises an isolating valve selectively and alternately operable in an isolating state isolating the second outlet port from the second inlet port, and in a communicating state communicating the second outlet port with the second inlet port for applying vacuum from the external vacuum source to the second outlet port, and advantageously, the pressure monitoring means is located in the insufflator, and preferably, the pressure monitoring means comprises a pressure sensor, and advantageously, the signal processor is programmed to operate the insufflator in the first operating mode as a default operating mode.

137. An insufflating system for insufflating a cavity in the body of a human or animal subject, the insufflating system comprising an insufflator comprising a first control means for controlling the supply of insufflating gas to a cavity in the body of a subject for insufflating the cavity to a set pressure, and a second control means for applying a vacuum to the cavity, an endoscope comprising a channel capable of accommodating an insufflating gas to the cavity of the subject, and a connecting means connecting the insufflator to the said channel of the endoscope for alternately applying insufflating gas and a vacuum to the cavity through the said channel of the endoscope.

138. An insufflating system as claimed in claim 137 in which the first control means and the second control means are configured for sequentially supplying insufflating gas and applying the vacuum to the said channel of the endoscope, and preferably, the first control means and the second control means are configured to supply insufflating gas and to apply the vacuum in sequential insufflating gas/vacuum cycles to the said channel of the endoscope, and advantageously, the first control means is configured for supplying insufflating gas to the said channel of the endoscope during each insufflating gas/vacuum cycle for a predefined gas supply time period, and the second control means is configured for applying the vacuum to the said channel of the endoscope during each insufflating gas/vacuum cycle for a predefined vacuum application time period, and preferably, each predefined gas supply time period lies in the range of 4 seconds to 10 seconds, and advantageously, each predefined vacuum application time period lies in the range of 1 second to 5 seconds, and preferably, the duration of each predefined vacuum application time period lies in the range of one eighth to one half of the duration of one insufflating gas/vacuum cycle, and advantageously, the duration of each predefined vacuum application time period is approximately one third of the duration of one insufflating gas/vacuum cycle.

139. An insufflating system as claimed in claim 137 in which the second control means is responsive to the cavity pressure exceeding the set pressure or a predefined upper pressure greater than the set pressure for applying the vacuum to the said channel of the endoscope, and preferably, the second control means is responsive to the cavity pressure exceeding the preset pressure or the predefined upper pressure for applying the vacuum to the said channel of the endoscope for one predefined vacuum application time period, and advantageously, the second control means is configured to apply the vacuum to the said channel of the endoscope until the cavity pressure is reduced to or below the set pressure or to a predefined lower pressure below the set pressure, and preferably, the connecting means comprises a first port being configured for communicating with the said channel of the endoscope, a second port being configured for communicating with the first control means, and a third port being configured for communicating with the second control means, and preferably, the connecting means comprises a Y-connector, and advantageously, the insufflator comprises a first outlet port and a second outlet port, and the first control means communicates with the first outlet port, and the second control means communicates with the second outlet port, and preferably, the second port of the connecting means is configured for communicating with the first outlet port, and the third port of the connecting means is configured for communicating with the second outlet port, and advantageously, the insufflator comprises a pressure monitoring means adapted for selectively connecting to the said channel of the endoscope or to another channel of the endoscope for monitoring the pressure in the cavity, and preferably, the channel of the endoscope capable of accommodating insufflating gas to the cavity of a subject comprises one of an instrument channel of the endoscope, an insufflating channel of the endoscope, a vacuum channel of the endoscope, or a channel defined by a tubular member attached and extending to the endoscope externally thereof, and advantageously, the vacuum applied to the cavity by the second control means is adapted for drawing smoke and/or gases from the cavity, and preferably, the first control means is configured to apply insufflating gas to the cavity for maintaining the cavity pressure at the set pressure, and advantageously, the insufflator is adapted for connecting to an external source of insufflating gas, and the first control means is configured for connecting to the external source of insufflating gas, and preferably, the insufflator is adapted for connecting to an external vacuum source, and the second control means is configured for connecting to the external vacuum source.

140. An insufflating system for insufflating a cavity in the body of a human or animal subject, the insufflating system comprising the insufflator as claimed in claim 131, and an endoscope comprising a channel capable of accommodating an insufflating gas to the cavity of the subject, and a connecting means connecting the insufflator to the said channel for alternately applying the insufflating gas and the vacuum to the said channel of the endoscope.

141. An insufflating system as claimed in claim 140 in which the connecting means is adapted for connecting to the first control means and to the second control means and to the said channel of the endoscope, and preferably, the connecting means comprises a first port being configured for communicating with the said channel of the endoscope, a second port being configured for communicating with the first control means, and a third port configured for communicating with the second control means, and advantageously, the second port of the connecting means is adapted for coupling to the first outlet port of the insufflator, and the third port of the connecting means is adapted for coupling to the second outlet port of the insufflator, and preferably, the connecting means comprises a Y-connector, and advantageously, the said channel of the endoscope capable of accommodating insufflating gas to the cavity of a subject comprises one of an instrument channel of the endoscope, an insufflating gas channel of the endoscope, a vacuum channel of the endoscope, or a channel extending through a tubular member attached to the endoscope and extending externally along the endoscope.

142. An insufflator for insufflating a cavity in the body of a human or animal subject, the insufflator comprising a first control means for controlling the supply of insufflating gas to the cavity, a second control means for controlling application of a vacuum to the cavity, and a signal processor programmed to sequentially and alternately operate the first control means to supply insufflating gas to the cavity, and the second control means to apply a vacuum to the cavity in a plurality of sequential pressure/vacuum cycles for maintaining an intermittent flow of insufflating gas through the cavity to withdraw smoke and/or other undesirable gases from the cavity.

143. An insufflator as claimed in claim 142 in which the signal processor is programmed to operate the first control means to supply insufflating gas to the cavity for a predefined gas supply time period in each pressure/vacuum cycle, and preferably, the signal processor operates the second control means to apply vacuum to the cavity for a predefined vacuum application time period in each pressure/vacuum cycle, and advantageously, each predefined vacuum application time period lies in the range of 1 second to 5 seconds, and preferably, the time duration of the predefined vacuum application time period of each pressure/vacuum cycle lies in the range of one eighth to one half of the duration of each pressure/vacuum cycle, and advantageously, the time duration of the predefined vacuum application time period is approximately one third of the duration of each pressure/vacuum cycle, and preferably, the signal processor is programmed to operate the first control means to prevent insufflating gas being supplied to the cavity during each predefined vacuum application time period of each pressure/vacuum cycle, and advantageously, each predefined gas supply time period lies in the range of 4 seconds to 10 seconds, and preferably, the signal processor is programmed to operate the second control means to prevent vacuum being applied to the cavity during each predefined gas supply time period of each pressure/vacuum cycle.

144. A method for insufflating a cavity in the body of a human or animal subject with an insufflator operable in selectable first and second operating modes, the method comprising monitoring the pressure in the cavity (cavity pressure), operating the insufflator in the first operating mode for insufflating the cavity to maintain the cavity pressure at a set pressure, and in response to the cavity pressure exceeding the set pressure or a predefined upper pressure greater than the set pressure operating the insufflator to apply a vacuum to the cavity until the cavity pressure falls to the set pressure, or operating the insufflator to apply a vacuum to the cavity for a first predefined time period, and operating the insufflator in the second operating mode for sequentially and alternately supplying insufflating gas to the cavity and applying a vacuum to the cavity in a plurality of sequential pressure/vacuum cycles.

145. A method as claimed in claim 144 in which the first predefined time period lies in the range of 1 second to 5 seconds, and preferably, on failure of the cavity pressure to fall to or below the set pressure at the end of the first predefined time period, the insufflator is operated to apply the vacuum to the cavity for at least one second time period, and advantageously, on failure of the cavity pressure to fall to or below the set pressure at the end of each second time period the insufflator is operated to apply the vacuum to the cavity for another second time period, and preferably, the time duration of the first one of the second time periods is computed as a function of the difference between the set pressure and the cavity pressure at the end of the first predefined time period, and advantageously, the time duration of the first one of the second time periods is computed as a function of the rate at which the cavity pressure dropped during the first predefined time period, and preferably, the time duration of the second and each subsequent one of the second time periods is computed as a function of the difference between the set pressure and the cavity pressure at the end of the previous one of the second time periods, and advantageously, the time duration of the second and each subsequent one of the second time periods is computed as a function of the rate at which the cavity pressure dropped during the previous one of the second time periods, and preferably, the time duration of each second time period is shorter than the time duration of the first predefined time period, and advantageously, the time duration of each subsequent second time period is shorter than the time duration of the immediately previous second time period, alternatively, the time duration of each one of the second time periods is the same as the time duration of the first predefined time period, and advantageously, the application of the vacuum to the cavity is terminated at the end of each one of the second time periods, and the cavity pressure is monitored, and preferably, the application of the vacuum to the cavity is terminated at the end of the first predefined time period and the cavity pressure is monitored.

146. A method as claimed in claim 144 in which in the first operating mode on the cavity pressure exceeding the set pressure or the predefined upper pressure, the supply of insufflating gas to the cavity is terminated until the cavity pressure falls to or below the set pressure, and preferably, the predefined upper pressure lies in the range of 0.5 mmHg to 5 mmHg above the set pressure, and advantageously, in the second operating mode, the cavity pressure is maintained within a predefined pressure range about the set pressure during each pressure/vacuum cycle, and preferably, the predefined pressure range about the set pressure lies in the range of 3 mmHg above the set pressure and 3 mmHg below the set pressure, and advantageously, insufflating gas is supplied to the cavity during each pressure/vacuum cycle for a predefined gas supply time period, and preferably, vacuum is applied to the cavity during each pressure/vacuum cycle for a predefined vacuum application time period, and advantageously, each predefined vacuum application time period lies in the range of 1 second to 5 seconds, and preferably, the duration of the predefined vacuum application time period lies in the range of one eighth to one half of the duration of each pressure/vacuum cycle, and advantageously, the duration of the predefined vacuum application time period is approximately one third of the duration of each pressure/vacuum cycle, and preferably, the supply of insufflating gas to the cavity is interrupted during the predefined vacuum application time period of each pressure/vacuum cycle, and preferably, each predefined gas supply time period lies in the range of 4 seconds to 10 seconds, and advantageously, the application of vacuum to the cavity is interrupted during the predefined gas supply time period of each pressure/vacuum cycle.

147. A method as claimed in claim 144 in which in the first operating mode the application of vacuum to the cavity is prevented when the first insufflating gas is being supplied to the cavity, and preferably, in the first operating mode the supply of insufflating gas to the cavity is prevented when vacuum is being supplied to the cavity, and advantageously, in the first operating mode on the cavity pressure falling to or below the set pressure insufflating gas is supplied to the cavity for maintaining the cavity pressure at the set pressure, and preferably, application of vacuum to the cavity is terminated on the cavity pressure falling to our below a predefined lower pressure lower than the set pressure, and insufflating gas is supplied to the cavity until the cavity pressure returns to the set pressure, and advantageously, the predefined lower pressure lies in the range of 2 mmHg to 8 mmHg below the set pressure, and preferably, the set pressure lies in the range of 2 mmHg to 20 mmHg, and preferably, is selectable, and advantageously, the signal processor is operated in the first operating mode as a default operating mode.

148. A method as claimed in claim 144 in which insufflating gas is supplied to the cavity and the vacuum is applied to the cavity from the insufflator through a channel in an endoscope capable of accommodating insufflating gas to the cavity, and preferably, the said channel of the endoscope is connected to a first control means of the insufflator which controls the supply of insufflating gas to the cavity for accommodating insufflating gas to the cavity, and advantageously, the said channel of the endoscope is connected to a second control means of the insufflator which controls the application of the vacuum to the cavity for applying the vacuum to the cavity, and preferably, the said channel of the endoscope is connected to the first and second control means through a connecting means, and advantageously, the connecting means comprises a first port communicating with the said channel of the endoscope, a second port communicating with the first control means, and a third port communicating with the second control means, and preferably, the connecting means comprises a Y-connector, and advantageously, the said channel of the endoscope capable of accommodating insufflating gas to the cavity of a subject comprises one of an instrument channel of the endoscope, an insufflating gas channel of the endoscope, a vacuum channel of the endoscope, or a channel extending through a tubular member attached to the endoscope and extending externally along the endoscope, and preferably, the cavity pressure is monitored through the said channel of the endoscope, and preferably, the cavity pressure is monitored through a channel of the endoscope other than the said channel, and advantageously, the endoscope is inserted into the cavity through a bodily orifice of the subject, and preferably, gases drawn from the cavity during application of vacuum to the cavity are filtered, and advantageously, the gases drawn from the cavity by the application of vacuum to the cavity are filtered through a bacterial-viral filter, and preferably, the filter is connected between the said channel of the endoscope and the insufflator, and advantageously, water entrained in gases drawn from the cavity by the application of vacuum to the cavity is collected in a water collecting means, and preferably, the water collecting means is located between the said channel of the endoscope and the insufflator.

149. A method for insufflating a cavity in the body of a human or animal subject comprising sequentially and alternately supplying insufflating gas to the cavity and applying a vacuum to the cavity in a plurality of sequential pressure/vacuum cycles for maintaining an intermittent flow of insufflating gas through the cavity to withdraw smoke and/or other undesirable gases from the cavity.

150. A method for insufflating a cavity in the body of a human or animal subject and for controlling the pressure at which the cavity is insufflated substantially at a set pressure, the method comprising inserting an endoscope into the cavity, connecting a channel of the endoscope capable of accommodating an insufflating gas to the cavity to an insufflator, and sequentially and alternately applying insufflating gas to the said channel of the endoscope for delivery to the cavity and applying a vacuum to the said channel of the endoscope in sequential pressure/vacuum cycles for withdrawing the smoke or the gas from the cavity with the cavity maintained substantially at the set pressure.
